(19) United States
(12) Patent Application Publication
(10) Pub. No.: US 2022/0084657 A1
(43) Pub. Date: Mar. 17, 2022

(54) CARE RECORDING DEVICE, CARE RECORDING SYSTEM, CARE RECORDING PROGRAM, AND CARE RECORDING METHOD

(71) Applicant: SUNCREER CO.,LTD., Sapporo-shi, Hokkaido (JP)

(72) Inventor: Masato MORI, Sapporo-shi, Hokkaido (JP)

(21) Appl. No.: 17/421,513

(22) PCT Filed: Jan. 10, 2020
(86) PCT No.: PCT/JP2020/000636

§ 371 Date: Jul. 8, 2021

(30) Foreign Application Priority Data
Jan. 11, 2019 (JP) 2019-003873

(51) Int. Cl. G16H 30/20; G16H 40/63

(52) U.S. Cl. CPC G16H 30/20; G16H 40/63

(57) ABSTRACT

A care recording device has an imaging data acquisition unit 341 that acquires imaging data from a camera capturing an image of the care recipient; a person determination unit 342 that detects a person imaged, to determine whether the person is the care recipient or the caregiver, a care recipient state determination unit 343 that determines a state type of the care recipient and stores, in a care history storage unit 337, care history information in which date and time are associated with the state type, and an assistive task determination unit 344 that, in a case where the person includes the caregiver, determines a type of the assistive task of the caregiver, associates the type of the assistive task with the care history information, and stores the association in the care history storage unit 337.

### TECHNICAL FIELD

The present invention relates to a care recording device, a care recording system, a care recording program, and a care recording method for performing recording related to care.

### BACKGROUND ART

In recent years, with the increase in the number of people requiring care due to the aging society and the decrease in the productive-age population, the care industry suffers a chronic labor shortage, and the amount of work per caregiver has been increasing. For this reason, technology has been developed to reduce the work burden on a caregiver as much as possible.

For example, in order to reduce the burden of patrol work and provide appropriate care for a care recipient, a watching system has been developed in which a surveillance camera is installed in each of rooms where the care recipient lives, to monitor and record activities of the care recipient in the form of videos or the like. However, a problem of this watching system where surveillance cameras are used is that, since the videos filmed by the surveillance cameras are recorded as-is, the privacy of the care recipient cannot be protected. Moreover, the videos captured by the surveillance cameras are displayed in real-time on a surveillance monitor or the like so that the care recipient can be monitored for danger. However, a caregiver is typically preoccupied with caregiving work, making it difficult for the caregiver to monitor the care recipient through the surveillance monitor, thereby taking long to notice a dangerous state of the care recipient.

Watching systems that use various sensors in place of the surveillance cameras described above have been developed so far. For example, Patent Literature 1 discloses an information processing device that acquires and displays time series data obtained by recording a state of sleep, an activity and the like of a care recipient from a sleep sensor, a motion sensor, a bathroom sensor, and the like.

Also, for the purpose of sharing information between caregivers and providing information to family members, the caregivers record assistive tasks and the like provided for the care recipients. However, such recording and sharing of information on top of daily busy operations such as assistive tasks are a heavy burden for the caregivers. Therefore, a technique has been developed by the caregivers to support the recording work for recording assistive tasks. Patent Literature 2, for example, discloses a program for use in a care recording mobile terminal that is carried by caregiving staff who provides care for a care subject, and records, in a predetermined format, results of the care provided for the care subject by the caregiving staff.

### CITATION LIST

### Patent Literature

### Patent Literature 1

Japanese Patent Laid-Open No. 2017-174012

### Patent Literature 2

Japanese Patent Laid-Open No. 2016-85673

### SUMMARY OF INVENTION

### Technical Problem

However, in the watching system of Patent Literature 1 that uses various sensors, although activities of a care recipient can be recorded and then verified, meal assistance, bathroom assistance, bathing assistance and other assistive tasks provided for the care recipient cannot be recorded. Even if the assistive tasks are recorded using the various sensors described above, a dedicated sensor needs to be installed for each type of assistive task, which makes the system extremely complicated and costly.

In the care recording method of Patent Literature 2 that uses a care recording mobile terminal, every time the caregiver provides an assistive task, the caregiver has to operate a screen displayed on the care recording mobile terminal to make a predetermined input, which is extremely troublesome. If the caregiver is not familiar with operating the care recording mobile terminal, such complicated operation of the care recording mobile terminal adds to the burden of the caregiver.

The present invention has been made to solve the foregoing problems, and an object of the present invention is to provide a care recording device, a care recording system, a care recording program, and a care recording method that are capable of achieving an improved efficiency of caregiving work with an inexpensive, simple system configuration by performing recording related to living conditions of care recipients and recording related to assistive tasks of caregivers simultaneously and easily.

### Solution to Problem

In order to accomplish the foregoing object of achieving an improved efficiency of caregiving work with an inexpensive, simple system configuration by performing recording related to lives of care recipients and recording related to assistive tasks of caregivers simultaneously and easily, a care recording device according to the present invention is a care recording device for recording a state of a care recipient and an assistive task provided for the care recipient by a caregiver, the care recording device including: an imaging data acquisition unit that acquires imaging data from a camera capturing an image of the care recipient; a person determination unit that detects a person imaged, based on the imaging data, to determine whether the person is the care recipient or the caregiver; a care recipient state determination unit that determines a state type of the care recipient based on the imaging data and stores, in a care history storage unit, care history information in which date and time are associated with the state type; and an assistive task determination unit that, in a case where the person determined by the person determination unit includes the caregiver, determines a type of the assistive task of the caregiver based on the imaging data, associates the type of the assistive task with the care history information, and stores the association in the care history storage unit.

According to one aspect of the present invention, in order to accomplish an object of achieving an improved efficiency of determining the state type of the care recipient from the imaging data imaging the care recipient and recording the living condition of the care recipient, the care recipient state determination unit may have a posture determination mode for detecting each coordinate of a body part representing a posture of the care recipient from the imaging data, and determining the state type of the care recipient based on the each coordinate of the body part.

According to one aspect of the present invention, in order to accomplish the object of achieving an improved efficiency of determining the state type of the care recipient from the imaging data imaging the care recipient and recording the living condition of the care recipient, the care recipient state determination unit may have a state learning determination mode for determining the state type based on state learned data obtained by learning the imaging data obtained in advance for each state of the care recipient and the imaging data acquired.

According to one aspect of the present invention, in order to accomplish an object of achieving an improved efficiency of determining the state type of the care recipient from the imaging data imaging the care recipient and recording the living condition of the care recipient, and an object of accelerating the discovery of a dangerous state of the care recipient, the care recording device may include: an area setting unit that sets an area smaller than an imaging range of the imaging data within the imaging range; and a body part setting unit that sets the body part of the care recipient in association with the area set by the area setting unit, wherein the care recipient state determination unit may have a body part area determination mode that detects, from the imaging data, a position within the imaging range representing each of body parts of the care recipient, and determines the state type of the care recipient based on whether or not a position of a body part set by the body part setting unit out of positions of the body parts exists within the area set by the area setting unit.

According to one aspect of the present invention, in order to accomplish an object of reducing the burden on the caregiver who records an assistive task, the assistive task determination unit may have a gesture determination mode for detecting, from the imaging data, whether the caregiver makes a predetermined gesture or not, and when the gesture is detected, determining a type of the assistive task associated with the gesture.

In addition, according to one aspect of the present invention, in order to accomplish the object of reducing the burden on the caregiver who records an assistive task, the assistive task determination unit may have a finger determination mode for detecting, from the imaging data, whether the caregiver makes a finger raising movement or not, and when the finger raising movement is detected, determining a type of the assistive task associated with the number of fingers raised.

According to one aspect of the present invention, in order to accomplish the object of further reducing the burden on the caregiver by automatically recording types of assistive tasks, and an object of preventing the occurrence of record omission caused by forgetting to record, the assistive task determination unit may have an assistive task learning determination mode for determining a type of the assistive task based on assistive task learned data acquired by learning the imaging data obtained in advance for each type of the assistive task, and the imaging data acquired.

According to one aspect of the present invention, in order to accomplish an object of easily and efficiently displaying a daily life of the care recipient, the care recording device may include a care recording image generation unit that uses the care history information stored in the care history storage unit to generate a care recording image for displaying the state type of the care recipient and the type of the assistive task side by side on a same screen, for each care recipient.

According to one aspect of the present invention, in order to accomplish an object of detecting an abnormality of the care recipient based on a physical evaluation level defined for each care recipient and notifying the resultant abnormal state, the care recording device may include: a physical evaluation level storage unit that stores a physical evaluation level defined for each care recipient; an abnormal state assessment unit that assesses whether or not a state type of a target care recipient determined by the care recipient state determination unit is an abnormal state corresponding to the physical evaluation level of the care recipient; and an abnormal state notification unit that, when the care recipient is assessed to be in an abnormal state, notifies that the care recipient is in the abnormal state.

In order to accomplish the object of achieving an improved efficiency of caregiving work with an inexpensive, simple system configuration by performing recording related to lives of care recipients and recording related to assistive tasks of caregivers simultaneously and easily, a care recording system according to the present invention includes: the care recording device; and a care recording camera that is installed in a living room of the care recipient, captures an image of the room, and transmits resultant imaging data to the care recording device.

In order to accomplish the object of achieving an improved efficiency of caregiving work with an inexpensive, simple system configuration by performing recording related to lives of care recipients and recording related to assistive tasks of caregivers simultaneously and easily, a care recording program according to the present invention is a care recording program for recording a state of a care recipient and an assistive task provided for the care recipient by a caregiver, the care recording program causing a computer to function as: an imaging data acquisition unit that acquires imaging data from a camera capturing an image of the care recipient; a person determination unit that detects a person imaged, based on the imaging data, to determine whether the person is the care recipient or the caregiver; a care recipient state determination unit that determines a state type of the care recipient based on the imaging data and stores, in a care history storage unit, care history information in which date and time are associated with the state type; and an assistive task determination unit that, in a case where the person determined by the person determination unit includes the caregiver, determines a type of the assistive task of the caregiver based on the imaging data, associates the type of the assistive task with the care history information, and stores the association in the care history storage unit.

In order to accomplish the object of achieving an improved efficiency of caregiving work with an inexpensive, simple system configuration by performing recording related to lives of care recipients and recording related to assistive tasks of caregivers simultaneously and easily, a care recording method according to the present invention is a care recording method for recording a state of a care recipient and an assistive task provided for the care recipient by a caregiver, the care recording method including: an imaging data acquisition step of acquiring imaging data from a camera capturing an image of the care recipient; a person determination step of detecting a person imaged, based on the imaging data, to determine whether the person is the care recipient or the caregiver; a care recipient state determination step of determining a state type of the care recipient based on the imaging data and storing, in a care history storage unit, care history information in which date and time are associated with the state type; and an assistive task determination step of, in a case where the person determined by the person determination step includes the caregiver, determining a type of the assistive task of the caregiver based on the imaging data, associating the type of the assistive task with the care history information, and storing the association in the care history storage unit.

### Advantageous Effects of Invention

According to the present invention, an improved efficiency of caregiving work can be achieved with an inexpensive, simple system configuration by performing recording related to lives of care recipients and recording related to assistive tasks of caregivers simultaneously and easily.

### BRIEF DESCRIPTION OF DRAWINGS

FIG. 1 shows a block diagram showing an embodiment of a care recording system according to the present invention.

FIG. 2 shows a diagram showing examples of posture states out of state types of a care recipient according to the present embodiment.

FIG. 3 shows a diagram showing areas that are set within an imaging range of imaging data by an area setting unit according to the present embodiment.

FIG. 4 shows a diagram showing examples of how a physical evaluation level is easily set according to the present embodiment.

FIG. 5 shows a diagram showing an example of a care history table stored in a care history storage unit according to the present embodiment.

FIG. 6 shows a diagram showing a result of detecting coordinates of respective body parts from imaging data in a posture determination mode of a care recipient state determination unit according to the present embodiment.

FIG. 7 shows a diagram showing a result of detecting coordinates of respective body parts from imaging data which are subjected to area setting in a body part area determination mode of the care recipient state determination unit according to the present embodiment.

FIG. 8 shows a diagram showing an example of a care recording image generated by a care recording image generation unit according to the present embodiment.

FIG. 9 shows a diagram showing an example of an abnormality recording image generated by the care recording image generation unit according to the present embodiment.

FIG. 10 shows a diagram showing an example of displaying abnormality information notified by an abnormal state notification unit according to the present embodiment.

FIG. 11 shows a flowchart showing actions of a care recording system according to the present embodiment.

FIG. 12 shows a flowchart showing actions of a posture determination mode of the care recipient state determination unit according to the present embodiment.

FIG. 13 shows a flowchart showing actions of a state learning determination mode of the care recipient state determination unit according to the present embodiment.

FIG. 14 shows a flowchart showing actions of a body part area determination mode of the care recipient state determination unit according to the present embodiment.

FIG. 15 shows a flowchart showing actions of a gesture determination mode of an assistive task determination unit according to the present embodiment.

FIG. 16 shows a flowchart showing actions of a finger determination mode of the assistive task determination unit according to the present embodiment.

FIG. 17 shows a flowchart showing actions of an assistive task learning determination mode of the assistive task determination unit according to the present embodiment.

### DESCRIPTION OF EMBODIMENT

An embodiment of a care recording device, a care recording system, a care recording program, and a care recording method according to the present invention is now described hereinafter with reference to the drawings.

A care recording system 1 according to the present embodiment is for recording a living condition of a care recipient and an assistive task provided for the care recipient by a caregiver, and includes, in the present embodiment, a care recording camera 2 that captures images of a living room of a care recipient, and a care recording device 3 that records a care history based on imaging data transmitted from the care recording camera 2. Each of configurations is described hereinafter.

The care recording camera 2 is installed in the living room and a hallway of the care recipient, in front of an elevator, and the like, and captures still images or films videos of the living room or the like. The care recording camera 2 according to the present embodiment is configured to be able to communicate with communication means 31 of the care recording device 3 by a wired/wireless LAN, WiFi, Bluetooth (R) or the like, and to transmit the captured images as imaging data to the care recording device 3 in real-time. An installation location for the care recording camera 2 in the living room, the number of care recording cameras 2 to be installed, and the like are determined based on primary postures of the care recipient in the living room. For example, in a case where the care recipient is bedridden, the care recording camera 2 is installed in a place where images of the care recipient can be taken from an angle at which changes in the posture of the care recipient from the bedridden state can be easily recognized.

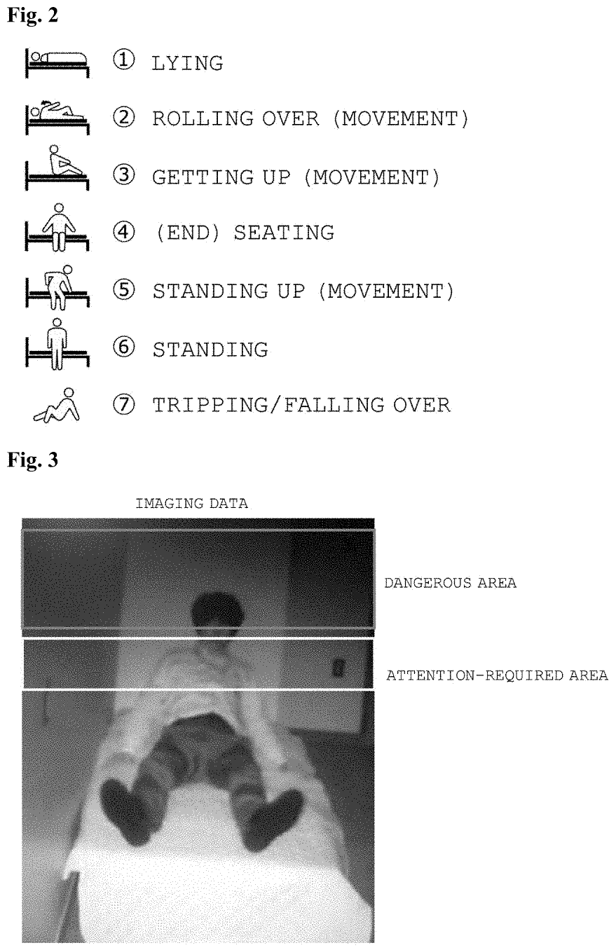

The care recording device 3 is for recording a state (state type) of the care recipient and an assistive task provided for the care recipient by the caregiver. In the present embodiment, the state types are various states in the life of the care recipient, and include various states that can be recorded in relation to the living conditions of the care recipient, such as sleeping. Specifically, as shown in FIG. 2, examples of the state types include states based on postures of the care recipient such as lying (sleeping), rolling over (movement), getting up (movement), (end) seating, standing up (movement), standing, tripping/falling over, and the like, and states requiring attention and dangerous states based on physical evaluations on the care recipient, which are described hereinafter. In addition, assistive tasks are tasks related to physical assistance provided for the care recipient in general, such as rushing in response to a nurse call, assisting in getting the care recipient up, meal assistance, bathroom assistance, changing diapers, bathing assistance, assistance in changing clothes, sleeping assistance, and the like.

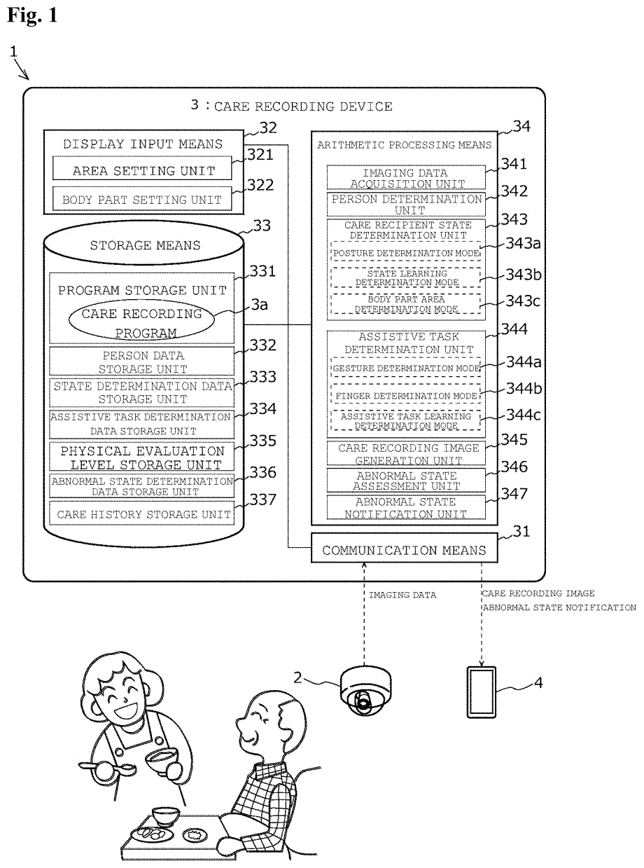

The care recording device 3 of the present embodiment is composed of computers such as a database server and mainly includes, as shown in FIG. 1, the communication means 31 communicating with the care recording camera 2, an external communication terminal 4 and the like, display input means 32 for displaying various display screens and inputting various data, storage means 33 for storing the various data and functioning as a working area for arithmetic processing means 34 to execute arithmetic processing, and the arithmetic processing means 34 for executing various types of arithmetic processing and functioning as each of components described hereinafter by executing a care recording program 3a installed in the storage means 33.

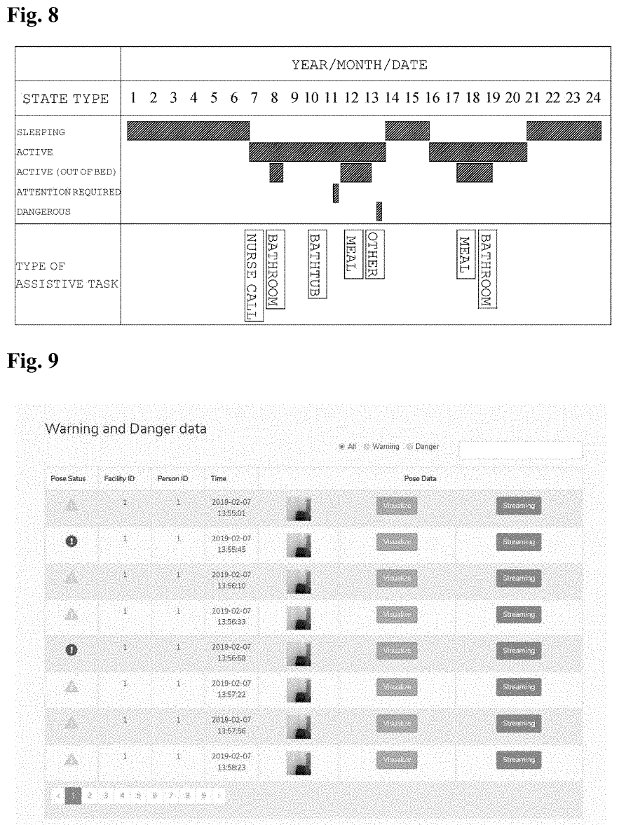

The communication means 31 is composed of a communication module and the like and is for implementing a communication function in the care recording device 3. The communication means 31 according to the present embodiment is configured to receive imaging data from the care recording camera 2 and transmit care recording images, abnormality recording images, abnormality notifications, and the like shown in FIGS. 8 to 10 to the external mobile terminal 4 and the like. Methods of communication are not particularly limited; examples thereof include wired/wireless LAN, WiFi, Bluetooth (R) and the like.

The display input means 32 is a user interface having an input function and a display function. The display input means 32 according to the present embodiment is composed of a display having a touch panel function and is mainly used as input means that functions as a monitor displaying various information, as well as an area setting unit 321 and a body part setting unit 322 that are described hereinafter. Note that the configuration of the display input means 32 is not limited to the touch panel display; the display input means 32 may separately include display means that only has a display function and input means such as a keyboard that only has an input function.

The storage means 33 is composed of a hard disk, a ROM (Read Only Memory), a RAM (Random Access Memory), a flash memory, and the like, and includes, as shown in FIG. 1, a program storage unit 331 storing the care recording program 3a, a person data storage unit 332 storing person data, a state determination data storage unit 333 storing state determination data used for determining a state of the care recipient, an assistive task determination data storage unit 334 storing assistive task determination data used for determining an assistive task of the caregiver, a physical evaluation level storage unit 335 storing a physical evaluation level for each care recipient, an abnormal state determination data storage unit 336 storing abnormal state determination data used for determining an abnormal state of the care recipient, and a care history storage unit 337 storing state types of the care recipients and assistive task types of the caregiver in chronological order.

The care recording program 3a for controlling the care recording device 3 of the present embodiment is installed in the program storage unit 331. The arithmetic processing means 34 executes the care recording program 3a to cause a computer to function as each of the components of the care recording device 3.

Forms of usage of the care recording program 3a are not limited to the configuration described above. For example, the care recording program 3a may be stored in a non-temporary recording medium that can be read by the computer, such as a CD-ROM or a USB memory, and may be read directly from the recording medium for execution. In addition, the care recording program 3a may be used by means of a cloud computing method, an ASP (Application Service Provider) method, or the like from an external server and the like.

The person data storage unit 332 is a database for storing person data used for determining a person. In the present embodiment, face recognition data that are obtained by imaging the face of a target person for use in face recognition processing by the person determination unit 342, personal information indicating whether the person is a care recipient or a caregiver, and an ID number for identifying the personal information, and the like, are stored in the person data storage unit 332. The person data are not limited to facial imaging data and the like, and may be appropriately selected from data that can be used for person determination processing by comparison with imaging data.

The state determination data storage unit 333 is for storing the state determination data used for determining a state of the care recipient. The state determination data storage unit 333 according to the present embodiment stores assessment coordinate data corresponding to the coordinates of a body part that are used for determining a state type of the care recipient by a posture determination mode 343a described hereinafter, state learned data used for determining a state type of the care recipient by a state learning determination mode 343b described hereinafter, and body part area determination data used for determining a state type of the care recipient by a body part area determination mode 343c described hereinafter.

Examples of the assessment coordinate data include coordinate values of each body part, relative coordinate values between body parts, and the like. The body parts include the head, the torso, an arm, a leg, a joint connecting body parts, and the like. In the present embodiment, coordinate data corresponding to body parts that enable assessment of a seated posture, a standing posture and the like of the care recipient are stored as the assessment coordinate data. The coordinate data stored as the assessment coordinate data are obtained by learning the coordinates of each body part that are extracted from an image of a seated posture or the like by using a posture estimation algorithm or the like used in the posture determination mode 343a which is described hereinafter. The image to be learned in this case is preferably taken in consideration of the physical evaluation level of the care recipient, the installation position of the care recording camera 2 in the living room, and the like, as described hereinafter. In a case where the imaging data use only an image captured from one direction, the coordinate values are based on two-dimensional coordinates obtained from the imaging data. Moreover, when using an image taken from multiple directions, not only the two-dimensional coordinates but also three-dimensional coordinates can be used.

The state learned data are obtained by learning the imaging data that are obtained by previously capturing an image of each state of the care recipient. In the present embodiment, the state learned data obtained by learning the imaging data capturing an image of the seated posture, standing posture or the like of the care recipient are stored.

The body part area determination data are composed of area data set by the area setting unit 321 used for determination by the body part area determination mode 343c, and body part data set by the body part setting unit 322.

The area setting unit 321 mainly sets an area within an imaging range for the imaging data in accordance with the physical evaluation level and the like of the care recipient, and in the present embodiment, the area setting unit 321 is configured by causing the display input means 32 to function as the input means. As shown in FIG. 3, the area to be set has an area narrower than the imaging range, and in the present embodiment, the area is stored as the coordinate data indicating a specific range within the imaging range of the imaging data. The area setting unit 321 according to the present embodiment is configured to be able to set a plurality of areas for one imaging range. Specifically, an attention-required area that is set to determine whether or not the state of the care recipient requires attention, and a dangerous area that is set to determine whether or not the state of the care recipient is a dangerous state, can be appropriately selected and set. In this manner, determination results can be divided with respect to the areas.

The body part setting unit 322 is for setting body parts of the care recipient in association with the areas set by the area setting unit 321. In the present embodiment, the body part setting unit 322 is configured by causing the display input means 32 to function as the input means. The body parts to be set include the head, the torso, an arm, a leg, a joint connecting body parts, and the like, and the name of a body part that is input by the display input means 32 using a keyboard, and the name of a body part that is selected and input from a plurality of names of body parts displayed, are stored using text data or the like.

The assistive task determination data storage unit 334 is for storing assistive task determination data used for determining an assistive task performed on the care recipient by the caregiver. The assistive task determination data storage unit 334 according to the present embodiment stores gesture data used for determination of an assistive task type of the caregiver by a gesture determination mode 344a described hereinafter, finger data used for determination of an assistive task type of the caregiver by a finger determination mode 344b described hereinafter, and assistive task learned data used for determination of an assistive task type of the caregiver by an assistive task learning determination mode 344c described hereinafter.

The gesture data are data associating a gesture made by the caregiver with an assistive task performed by the caregiver. The gesture is a movement or shape of a characteristic part of the caregiver. Examples of the gesture include a dynamic gesture that involves a movement depicting a geometric pattern such as a triangle, a square, or a star drawn with a fingertip or an arm, and a static gesture that does not involve a movement, such as a rock, scissors or paper. The gesture data are stored as the association between these gestures and the types of assistive tasks such as rushing in response to a nurse call, assisting in getting the care recipient up, meal assistance, bathroom assistance, changing diapers, bathing assistance, assistance in changing clothes, sleeping assistance, and the like.

The finger data are data in which the number of fingers protruding from a hand raised by the caregiver is associated with an assistive task. For example, one raised finger means rushing in response to a nurse call, and two raised fingers means assisting in getting the care recipient up. In this manner, the association between the number of fingers and the type of the assistive task is stored.

The assistive task learned data are obtained by learning the imaging data obtained by previously capturing an image of each assistive task of the caregiver. In the present embodiment, the assistive task learned data that are obtained by learning the imaging data obtained by capturing an image of a state of each assistive task type, are stored. For example, as shown in FIG. 1, by learning the imaging data obtained by capturing an image of a state of meal assistance, the assistive task learned data corresponding to the meal assistance are created and stored.

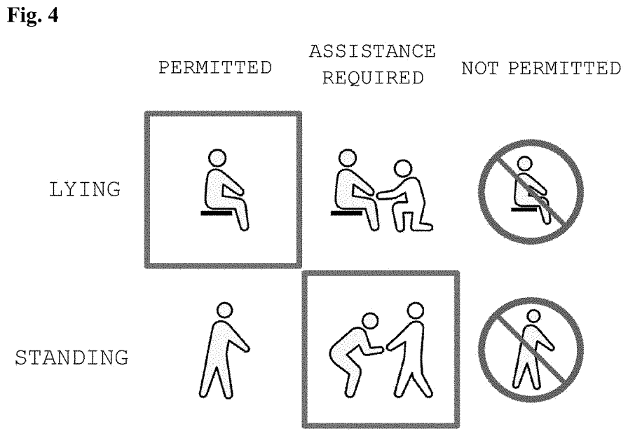

The physical evaluation level storage unit 335 is a database for storing a physical evaluation level of each care recipient. The physical evaluation level in the present embodiment are defined based on daily activities of the respective care recipients and results of manual muscle strength measurement. In the present embodiment, the physical evaluation levels are used for determining a normal state and an abnormal state in accordance with the posture of each care recipient determined by the posture determination mode 343a and the state learning determination mode 343b. As shown in FIG. 4, the physical evaluation level is defined as “permitted,” “assistance required,” or “not permitted” for a seated posture and a standing posture. Specifically, “permitted” in the seated posture is defined as a level at which the seated posture is always allowed, “assistance required” is defined as a level at which the seated posture is allowed under the circumstances where the care recipient is attended by the caregiver or has an assistive device, and “not permitted” is defined as a level at which the seated posture is not allowed. On the other hand, “permitted” in the standing posture is defined as a level at which the standing posture is always allowed, “assistance required” is defined as a level at which the standing posture is allowed under the circumstances where the care recipient is attended by the caregiver or has an assistive device, and “not permitted” is defined as a level at which the standing posture is not allowed. The evaluation level shown in FIG. 4 shows a level at which the seated posture is allowed freely but assistance is required in the standing posture.

In addition, examples of determining the physical evaluation levels include FIM (Functional Independence Measure), which is an international evaluation method for activities of daily life (ADL), and a method using MMT (manual muscle test) that is mainly used clinically to evaluate the muscle strengths of the whole body. FIM is an evaluation method based mainly on functional levels and includes six levels, “0. completely independent,” “1. independent in a special environment,” “2. light assistance,” “3. moderate assistance,” “4. intense assistance,” and “5. total assistance.”

MMT includes six evaluation levels where the level of the strength of a muscle that can be completely moved even under a strong resistance is evaluated as “5. normal,” the level of the strength of a muscle that can be completely moved even under a considerably strong resistance is evaluated as “4. good,” the level of the strength of a muscle that can be completely moved by overcoming the gravity is evaluated as “3. fair,” the level of the strength of a muscle that can be completely moved without the gravity is evaluated as “2. poor,” the level of the strength of a muscle that only shows muscle contraction without a joint movement is evaluated as “1. trace,” and the level of the strength of a muscle that does not show any muscle contraction is evaluated as “0. zero.”

In addition, four evaluations such as “0. perfect,” “1. partial,” “2. slight,” and “3. none” based on cognitive ability to eat, use a bathroom, or straighten the posture, and evaluation levels for best motion responses such as “0. complying with order,” “1. moving to painful area,” “2. avoiding limb flexion,” “3. abnormal limb flexion,” “4. stretching limb,” and “5. not moving at all.”

The physical evaluation level storage unit 335 stores, for each care recipient, the levels evaluated using at least any of the evaluation methods.

The abnormal state determination data storage unit 336 has a database of normal and abnormal states of the care recipient based on the physical evaluation level and the state type of the care recipient. In the present embodiment, a normal or abnormal state is stored for each combination of the physical evaluation level and the posture of the care recipient.

For example, as shown in FIG. 4, for the level at which the care recipient can sit freely but needs assistance when standing, when the state type indicates “seated posture,” “normal” is stored, but when the care recipient tries to stand up on his/her own or stood up from the seated posture, “abnormal” is stored. Similarly, when the physical evaluation level based on MMT indicates “5. normal” where the care recipient can move muscles without problems, and when the state type indicates “seated posture,” “normal” is stored. However, when the physical evaluation level based on MMT indicates “0. zero” where the care recipient cannot move muscles at all, “abnormal” is stored even if the state type indicates the same “seated posture,” because the care recipient cannot even stay in the seated position. In this manner, a normal/abnormal state with respect to the state type varies according to the physical evaluation level. Thus, the abnormal state determination data storage unit 336 stores a normal/abnormal state for the state type corresponding to each physical evaluation level. Assessment of a normal state and an abnormal state is not particularly limited, but abnormal state may be classified based on the levels of abnormalities, such as normal, slightly abnormal, abnormal, extremely abnormal. The number of categories of these abnormal levels, the corresponding names, and the like may be selected appropriately.

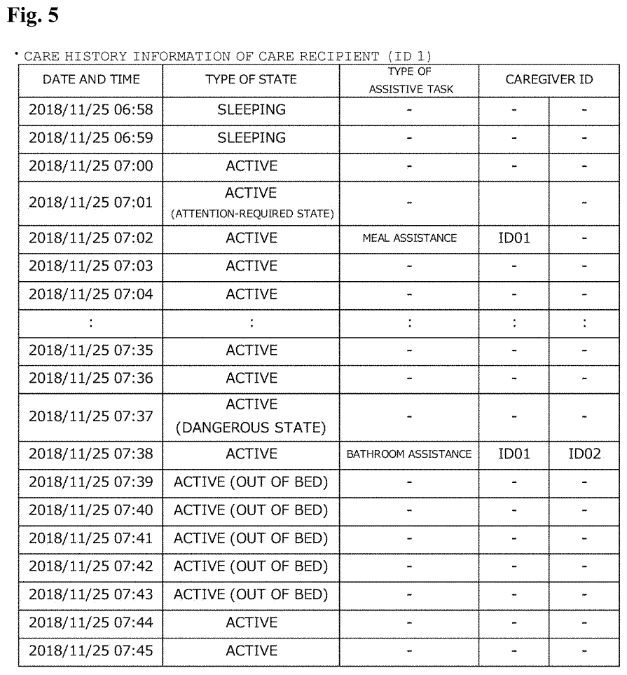

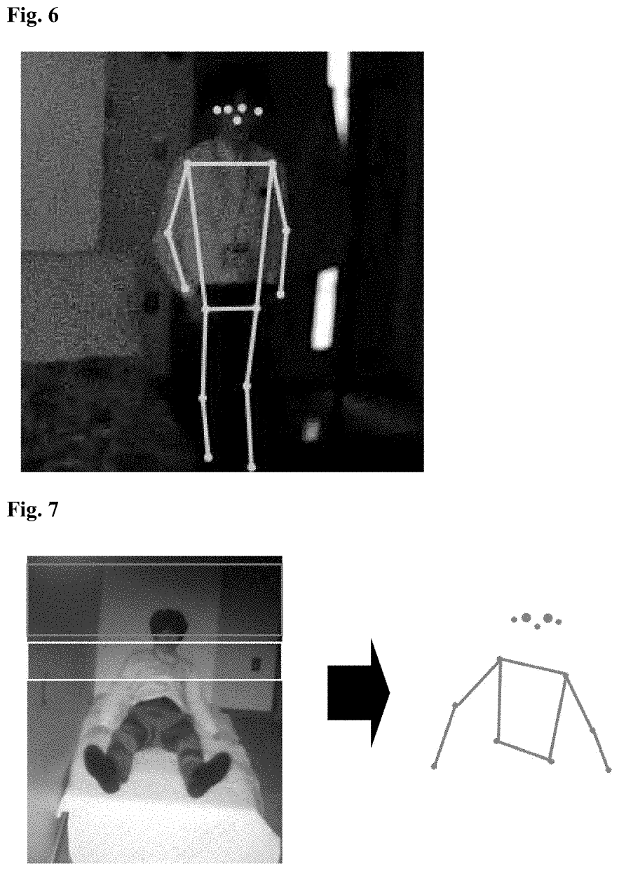

The care history storage unit 337 is for storing care history information in which information on the state of the care recipient and on an assistive task are accumulated for each care recipient (for each care recipient ID) in chronological order. As shown in FIG. 5, the care history storage unit 337 according to the present embodiment stores, at predetermined time intervals, the care history information in which date and time are associated with the state type determined by a care recipient state determination unit 343 described hereinafter, such as the state of a posture determined by the posture determination mode 343a or the state learning determination mode 343b, and a state requiring attention or a dangerous state determined by the body part area determination mode 343c. The care history storage unit 337 can also store the association between the type of an assistive task determined by the assistive task determination unit 344 and a caregiver ID of a person who performs the assistive task. Also, in the present embodiment, in a case where the determination result obtained by the body part area determination mode 343c indicates a state requiring attention or a dangerous state, the imaging data obtained by capturing an image of the state can be stored.

Note that items of the care history information stored in the care history storage unit 337 are not limited to those shown in FIG. 5, and may be increased or decreased, as necessary. Furthermore, in a case where an assistive task performed by the caregiver for the state of the care recipient can be associated mutually, the care history information of the care recipient and the care history information of the caregiver may be managed in separate storage units. In addition, the care history information may be stored as items separate from the state types such as a normal/abnormal state, a state requiring attention, or a dangerous state.

The arithmetic processing means 34 is described next. The arithmetic processing means 34 is composed of a CPU (Central Processing Unit) or the like, and, by the execution of the care recording program 3a installed in the storage means 33, functions as an imaging data acquisition unit 341, a person determination unit 342, a care recipient state determination unit 343, an assistive task determination unit 344, an assistance recording image generation unit 345, an abnormal state assessment unit 346, and an abnormal state notification unit 347, as shown in FIG. 1.

The imaging data acquisition unit 341 acquires imaging data transmitted from the care recording camera 2. In the present embodiment, the imaging data acquisition unit 341 acquires the imaging data transmitted from the care recording camera 2 via the communication means 31 at predetermined time intervals.

The person determination unit 342 detects a person captured in the imaging data acquired by the imaging data acquisition unit 341, and determines whether the person is a care recipient or a caregiver. Specifically, a general person detection algorithm is used to determine whether or not a person area is extracted in the imaging data. In a case where the person area is extracted, the face recognition data are read from the person data storage unit 332, and a facial image stored as the face recognition data is collated with a face area of the extracted person area, thereby determining whether the person is a care recipient or a caregiver.

Note that, in the present embodiment, whether the person is a care recipient or a caregiver is determined by face recognition, but the method for determining whether the person is a care recipient or a caregiver is not limited to the method using face recognition. For example, in a case where the position of the detected person is inside a bed area, the person may be determined to be a care recipient, and in a case where the position of the detected person is a predetermined position outside the bed area, the person may be determined to be a caregiver.

The care recipient state determination unit 343 determines a state type of the care recipient. In the present embodiment, the care recipient state determination unit 343 includes the posture determination mode 343a for determining the state type of the care recipient based on each coordinate of a body part, the state learning determination mode 343b for determining the state type based on the state learned data and the acquired imaging data, and the body part area determination mode 343c for determining whether the state of the care recipient is a state requiring attention or a dangerous state based on whether or not a specific body part is present in a set area.

As shown in FIG. 6, the posture determination mode 343a uses the posture estimation algorithm to detect each of the coordinates of a joint or each part of the face (eyes, ears, nose, and the like) of the care recipient from the imaging data acquired by the imaging data acquisition unit 341, compare the detected coordinates with the assessment coordinate data stored in the state determination data storage unit 333, and obtain a degree of similarity between the detected coordinates and the assessment coordinate data and a feature quantity indicating posture, thereby determining the type such as the seated posture or the standing posture of the care recipient. The posture estimation algorithm can be selected appropriately, and examples thereof include OpenPose (tf-pose-estimation, and the like) using Deep Learning, PoseNet, BodyPix, and the like. The posture estimation algorithm may be executed by the arithmetic processing means 34 of the care recording device 3, or by a web browser that uses another device equipped with image processing means or a machine learning library, TensorFlow, which is open sourced by Google.

Note that the coordinates obtained in the posture determination mode 343a are for specifying the positions within the imaging range of the imaging data, and, as described above, can be appropriately selected from two-dimensional coordinates or three-dimensional coordinates in accordance with an imaging direction for capturing an image and the number of imaging directions.

The state learning determination mode 343b determines the types of a state based on the state learned data and the imaging data. Specifically, an image feature quantity representing an image area of the care recipient is calculated from the imaging data. Then, the state learned data stored in the state determination data storage unit 333 are read out, and the degree of similarity between the calculated image feature quantity and each state learned data value is calculated. When a degree of similarity calculated to be equal to or greater than a predetermined threshold value exists in the state learned data, the type of the state learned data is determined as the type of the state of the care recipient. When there exist a plurality of degree of similarity equal to or greater than the predetermined threshold value, the large degree of similarity may be used to determine the state type of the care recipient.

The body part area determination mode 343c sets an area within the imaging range of the imaging data, and determines the type of the state based on the presence or absence of a body part of the care recipient that is set in this area. In the present embodiment, whether the state of the care recipient is normal, requires attention, or dangerous is determined based on the area set by the area setting unit 321 and the body part set by the body part setting unit 322, the area and the body part being stored as the body part area determination data in the state determination data storage unit 333.

The body part area determination mode 343c according to the present embodiment uses the posture estimation algorithm as with the posture determination mode 343a, to detect coordinates of a joint or each part of the face (eyes, ears, nose, and the like) of the care recipient from the imaging data acquired by the imaging data acquisition unit 341, and specify body parts of the care recipient from the arrangement and the like of the detected coordinates. Then, it is assessed whether the position (coordinates) of the body part matching the body part set by the body part setting unit 322 out of the body parts is inside or outside the area set by the area setting unit 321. In the present embodiment, when the set body part is assessed to be outside the area, the state of the care recipient is determined to be normal, and when the set body part is assessed to be inside the area, the state of the care recipient is determined to require attention or be dangerous. Contrary to the present embodiment, this determination criterion may be set as a criterion for determining that the state of the care recipient is a state requiring attention or a dangerous state when the body part is assessed to be outside the area.

It should be noted that the detection of the position within the imaging range representing each body part of the care recipient from the imaging data is not limited to the method based on the coordinates obtained with the posture estimation algorithm; for example, as in the state learning determination mode 343b, the state learned data obtained by learning the image of each body part may be compared with the imaging data to detect the positions from the degree of similarity between these data.

In the care recipient state determination unit 343 according to the present embodiment, in a case where the person is not detected by the person determination unit 342 or the person detected is anyone other than the care recipient in any of the posture determination mode 343a, the state learning determination mode 343b, and the body part area determination mode 343c, it is determined that the care recipient is not present in the living room and that the state type of the care recipient is “active (out of bed).”

Then, the care recipient state determination unit 343 stores the determined state type in the care history storage unit 337 as the care history information created in chronological order for each care recipient, as shown in FIG. 5.

The assistive task determination unit 344 is for determining the types of assistive tasks, and determines whether the person within imaging data who is assessed by the person determination unit 342 includes a caregiver or not. In a case where a caregiver is included, the type of an assistive task performed by the caregiver is determined based on the imaging data. The assistive task determination unit 344 according to the present embodiment includes the gesture determination mode 344a for determining the type of an assistive task based on a gesture made by the caregiver, the finger determination mode 344b for determining the type of an assistive task based on the number of fingers raised by the caregiver, and the assistive task learning determination mode 344c for determining the type of an assistive task based on the assistive task learned data and the acquired imaging data.

In the gesture determination mode 344a, in a case where whether the caregiver makes a gesture or not is detected from the imaging data, and when the gesture is detected, the type of the assistive task associated with the gesture is determined. Specifically, first, a characteristic part of the caregiver, such as a hand area or an arm area, is extracted from the imaging data. Then, the gesture data are read from the assistive task determination data storage unit 334 and compared with a movement or shape of the extracted characteristic part, and the degree of similarity between patterns is calculated, to determine whether or not there exists a gesture in which the degree of similarity is equal to or greater than the predetermined threshold value. In a case where a gesture with the predetermined threshold value or higher exists, the type of the assistive task associated with this gesture pattern is determined as the type of the assistive task performed by the caregiver. In a case where there exist a plurality of gesture patterns in which the degree of similarity are equal to or greater than the predetermined threshold value, the gesture pattern with a large degree of similarity may be determined as the type of the assistive task.

In the finger determination mode 344b, in a case where whether the caregiver rises a finger or not is detected from the imaging data, and when a movement for raising a finger is detected, the number of raised fingers is extracted, and the assistive task associated with the number of raised fingers is determined as the type of the assistive task performed by the caregiver. For example, the imaging data are binarized into the hand area of the caregiver and other areas. The number of protruding parts in the hand area is detected. Then, the detected number is taken as the number of fingers raised by the caregiver. Thereafter, from the finger data stored in the assistive task determination data storage unit 334, the assistive task associated with the number of fingers is extracted, and this assistive task is determined as the type of the assistive task performed by the caregiver.

Note that the method for detecting the number of raised fingers is not limited to the method for binarizing the imaging data into the hand area of the caregiver and other areas, and therefore may be selected appropriately from other algorithms and the like that enable extraction of the number of raised fingers.

In the assistive task learning determination mode 344c, the type of an assistive task is determined based on the assistive task learned data and the imaging data. Specifically, an image feature quantity is calculated from the imaging data. The image feature quantity to be calculated may be of the caregiver and/or the care recipient. Then, the assistive task learned data stored in the assistive task determination data storage unit 334 are read out, and the degree of similarity between the calculated image feature quantity and each assistive task learned data value is calculated. In a case where the assistive task learned data include a calculated degree similarity equal to or greater than the predetermined threshold value, the type of the relevant assistive task learned data is determined as the type of the assistive task performed by the caregiver. In a case where there exist a plurality of degree of similarity equal to or greater than the predetermined threshold value, the large degree of similarity may be determined as the type of the assistive task performed by the caregiver.

In the assistive task learning determination mode 344c, the types of assistive tasks can also be determined using a machine learning algorithm. In other words, in the assistive task determination data storage unit 334, the assistive task learned data may be stored as a learning parameter set in the machine learning algorithm, the assistive task learned data may be set in a learned model, and it may be determined whether or not an output result obtained by inputting the imaging data with respect to the learned model is equal to or greater than the predetermined threshold value. In this case, when the output result is equal to or greater than the predetermined threshold value, the type of an assistive task that is the output result is determined.

Moreover, in the present embodiment, in order to comply with a situation where a plurality of caregivers perform an assistive task, not only the imaging data showing a single caregiver performing the assistive task, but also the imaging data showing the plurality of caregivers performing the assistive task, may be used for learning. As with the posture estimation algorithm, the machine learning algorithm may be executed by the arithmetic processing means 34 of the care recording device 3 or by another device equipped with image processing means or a web browser.

Then, the assistive task determination unit 344 associates the type of the assistive task with the care history information of the care recipient and stores the association in the care history storage unit 337. In the present embodiment, as shown in FIG. 5, the caregiver ID of the caregiver who performs the assistive task is stored together with the type of the assistive task. In a case where the assistive task is performed by a plurality of caregivers, the caregiver IDs of the respective caregivers are stored.

The care recording image generation unit 345 is for generating a care recording image for displaying a care record and an abnormality recording image for displaying information recorded as an abnormal state of the care recipient. When the display input means 32 or the external communication terminal 4 instructs the care recording image generation unit 345 of the present embodiment to display the care record, the care recording image generation unit 345 reads the care history information from the care history storage unit 337, generates a care recording image, and transmits the care recording image to the display input means 32. In the care recording image, the state types of care recipients and the types of assistive tasks performed on the care recipients are described side by side on the same screen and displayed for the respective care recipients.

For example, as shown in FIG. 8, a state type display field is provided in which items corresponding to respective state types (“sleep,” “active,” “active (out of bed)”) are arranged vertically with respect to a horizontal axis showing every hour of 24 hours, so that a daily life of the care recipient can be visually understood at a glance. In this state type display field, each item is colored in a band shape and displayed in association with a time range of each state type. In addition, below the state type display field is provided an assistive task display field for displaying the types of assistive tasks in association with the times at which the assistive tasks are performed.

Moreover, in a case where the display input means 32 or the external communication terminal 4 instructs to display an abnormal state, the care history information is read from the care history storage unit 337, and an abnormality recording image consisting of a list of information necessary for displaying abnormal states is generated. Specifically, as shown in FIG. 9, an abnormality recording image consisting of a determination result of an abnormal state such as a state requiring attention or a dangerous state, a date and time when the abnormality is determined, and the imaging data obtained at that time, is generated. The generated abnormality recording image is then transmitted to the display input means 32.

Note that displaying the care recording image is not limited to displaying by means of the display input means 32; as shown in FIG. 1, the image may be transmitted to and displayed by the external communication terminal 4 connected communicably via communication means.

The abnormal state assessment unit 346 assesses whether the care recipient is in a normal state or an abnormal state, based on the physical evaluation level of the care recipient and the type of the state of the care recipient assessed from the imaging data. Specifically, the abnormal state assessment unit 346 acquires the type of the state of the care recipient (posture state) determined by the posture determination mode 343a and the state learning determination mode 343b of the care recipient state determination unit 343, and reads, from the physical evaluation level storage unit 335, the physical evaluation level given to the care recipient. Then, an abnormality level defined by the combination of the physical evaluation level of the care recipient and the type of state, such as normal, abnormal, slightly abnormal, abnormal, or very abnormal, is extracted from the database stored in the abnormal state determination data storage unit 336, and whether the state of the care recipient is normal or abnormal is assessed based on the extracted abnormality level.

Also, in a case where the body part area determination mode 343c assesses that the position of the body part matching the body part set by the body part setting unit 322 is located within an area set by the area setting unit 321, the abnormal state assessment unit 346 acquires, from the state determination data storage unit 333, information indicating whether the area is an attention-required area or a dangerous area. When the area is the attention-required area, the care recipient is assessed to be in a state requiring attention, and when the area is the dangerous area, the care recipient is assessed to be in a dangerous state.

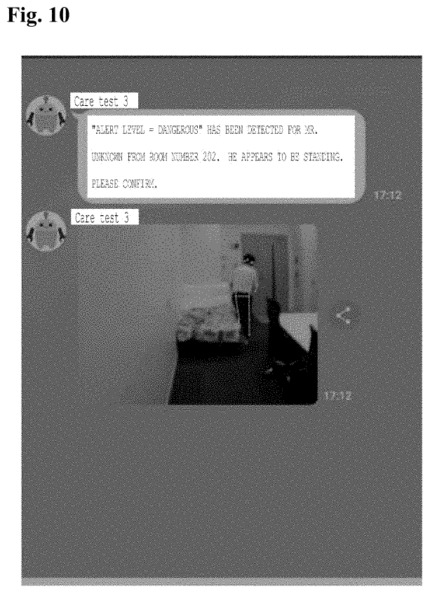

In a case where the abnormal state assessment unit 346 assesses that the state of the care recipient is an abnormal state and where the body part area determination mode 343c determines that the position of the body part matching the body part set by the body part setting unit 322 is located within the area set by the area setting unit 321, the abnormal state notification unit 347 sends a notification to that effect. In the present embodiment, the abnormal state and the imaging data of the care recipient in the abnormal state are transmitted to the display input means 32 or the external communication terminal 4 via the communication means 31. Accordingly, as shown in FIG. 10, a pop-up window for notifying the imaging data and the abnormal state of the care recipient can be displayed on the display screen of the display input means 32 or the external communication terminal 4, or a notification sound can be emitted from a speaker (not shown). A device that notifies the caregiver or the like of the abnormal state is not particularly limited, and may be appropriately selected from, for example, an emergency light (lamp) that can be turned on, flashed, turned off, or discolored according to the assessment result on the abnormal state.

Actions of each configuration in the care recording device 3, the care recording system 1, and the care recording program 3a according to the present embodiment and the care recording method are described next.

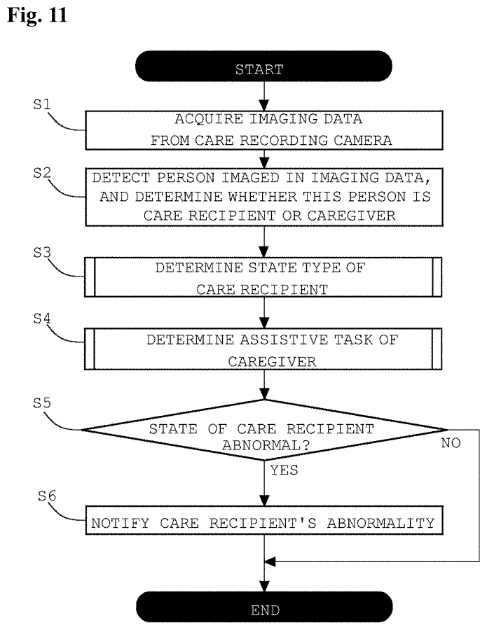

In the present embodiment, the care recording camera 2 captures images of the living room of the care recipient. As shown in FIG. 11, the imaging data acquisition unit 341 of the care recording device 3 acquires the imaging data of the living room of the care recipient captured by the care recording camera 2 via the communication means 31 (imaging data acquisition step: S1).

The person determination unit 342 detects a person captured in the imaging data and determines whether the person is a care recipient or a caregiver (person determination step: S2). This enables the distinction between the care recipient and the caregiver and the execution of appropriate determination processing for the care recipient and caregiver.

The care recipient state determination unit 343 determines the state type of the care recipient based on the imaging data (care recipient state determination step: S3). In the present embodiment, the care recipient state determination step is performed by one mode appropriately selected from the posture determination mode 343a, the state learning determination mode 343b, and the body part area determination mode 343c. Note that the number of modes to be selected is not limited to one; a plurality of modes may be selected to perform the determination step.

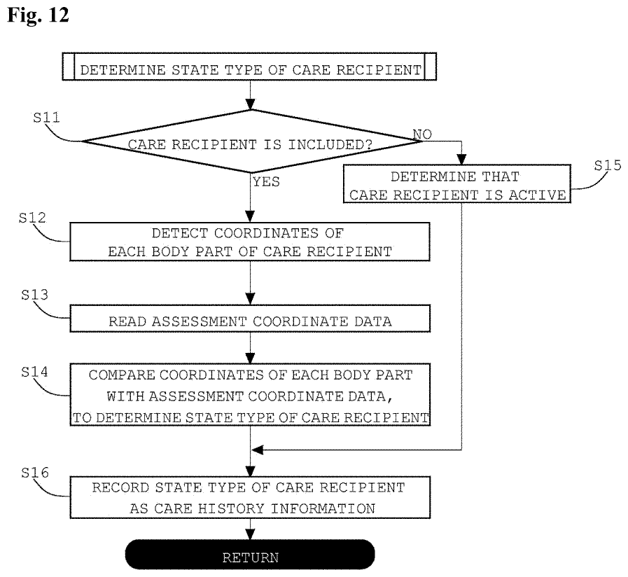

In a case where the posture determination mode 343a is selected, first, it is determined whether the person within the imaging data that is determined by the person determination unit 342 includes a care recipient or not, as shown in FIG. 12 (S11). In a case where a care recipient is included (S11: YES), coordinates of a joint or each part of the face (eyes, ears, nose, and the like) of the care recipient are detected from the imaging data acquired by the imaging data acquisition unit 341, and are stored in the storage means 33 (S12). Also, the assessment coordinate data are read from the state determination data storage unit 333 (S13). The detected coordinates are compared with the read assessment coordinate data to determine the state type of the care recipient (S14).

On the other hand, in a case where the person within the imaging data that is determined by the person determination unit 342 does not include a care recipient (S11: NO), it is determined that the person is outside the living room and active (“active (out of bed)”) (S15).

Then, the care recipient state determination unit 343 stores the determined state types of the care recipient in the care history storage unit 337 collectively in chronological order, as shown in FIG. 5 (S16).

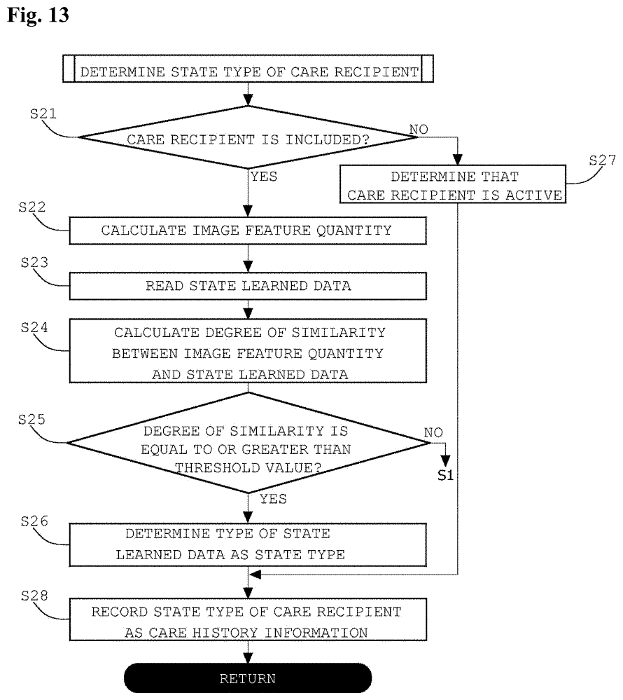

In a case where the state learning determination mode 343b is selected, it is determined whether the person within the imaging data that is determined by the person determination unit 342 includes a care recipient or not, as shown in FIG. 13 (S21), and when a care recipient is included (S21: YES), the image feature quantity representing an image area of the care recipient is calculated from the imaging data (S22). Also, the state learned data stored in the state determination data storage unit 333 are read (S23). Then, the degree of similarity between the calculated image feature quantity and each state learned data value is calculated (S24). Subsequently, it is assessed whether or not the calculated degree of similarity equal to or greater than the predetermined threshold value exists in the state learned data (S25). In a case where the degree of similarity equal to or greater than the predetermined threshold value exists (S25: YES), the type of this state learned data is determined as the type of the state of the care recipient (S26). In a case where the degree of similarity equal to or greater than the predetermined threshold value does not exist (S25: NO), the processing returns to S1 to attempt to determine the state type of the care recipient based on other imaging data.

On the other hand, in a case where the person within the imaging data that is determined by the person determination unit 342 does not include a care recipient (S21: NO), it is determined that the person is outside the living room and active (“active (out of bed)”) (S27).

Then, the determined state types of the care recipient are stored in the care history storage unit 337 collectively in chronological order (S28).

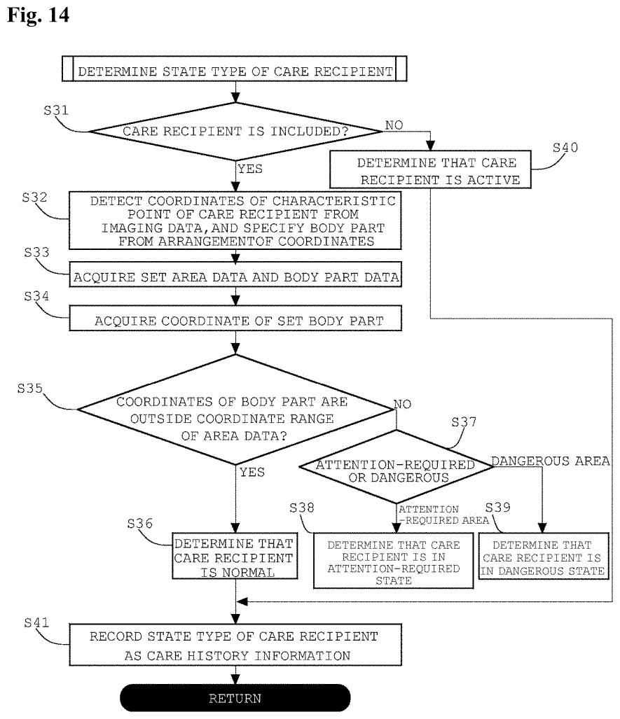

In a case where the body part area determination mode 343c is selected, it is determined whether the person within the imaging data that is determined by the person determination unit 342 includes a care recipient or not, as shown in FIG. 14 (S31). In a case where a care recipient is included (S31: YES), each coordinate of a joint or each part of the face (eyes, ears, nose, and the like) of the care recipient is detected from the imaging data acquired by the imaging data acquisition unit 341, and body parts of the care recipient are specified from the arrangement of the detected coordinates (S32). Next, area data and body part data stored as the body part area determination data are acquired from the state determination data storage unit 333 (S33). Then, the coordinates of the set body parts stored as the body part data are acquired based on the body parts of the care recipient that are specified in S32 (S34). Subsequently, it is assessed whether the coordinate positions of the body parts are outside the coordinate range of the area data or not (S35). In a case where the coordinates of the set body parts are outside the coordinate range of the area data (S35: YES), it is determined that the state of the care recipient is neither a state requiring attention nor a dangerous state and is thus normal (S36). On the other hand, in a case where the coordinates of the set body parts are not outside the coordinate range of the area data (are within the range) (S35: NO), the abnormal state assessment unit 346 assesses whether the area is an attention-required area or a dangerous area (S37). In a case where the area is set as the attention-required area (S37: attention-required area), the state type of the care recipient is determined as “state requiring attention” (S38). However, in a case where the area is set as the dangerous area (S37: dangerous area), the state type of the care recipient is determined as “dangerous state” (S39).

On the other hand, in a case where the person within the imaging data that is determined by the person determination unit 342 does not include a care recipient (S31: NO), it is determined that the person is outside the living room and active (“active (out of bed)”) (S40).

Then, the determined state types of the care recipient are stored in the care history storage unit 337 collectively in chronological order, as shown in FIG. 5 (S41).

In this manner, the care recipient state determination unit 343 stores the care history information in which the state type is associated with the date and time in the care history storage unit 337. Accordingly, since the imaging data do not need to be stored, the care history can be recorded while protecting the privacy of the care recipient.

Next, the assistive task determination unit 344 determines an assistive task performed by a caregiver (assistive task determination step: S4). In the present embodiment, the assistive task determination step is performed by one mode appropriately selected from the gesture determination mode 344a, the finger determination mode 344b, or the assistive task learning determination mode 344c. Note that the number of modes to be selected is not limited to one; a plurality of modes may be selected to perform the determination step.

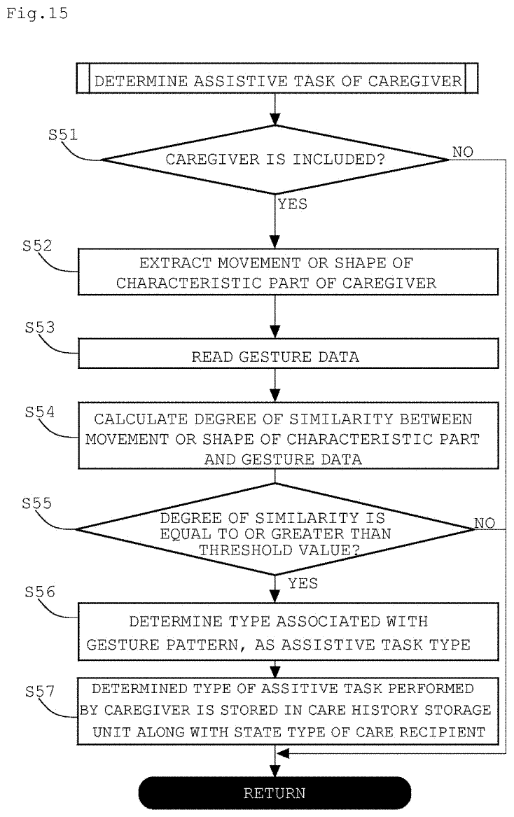

In a case where the gesture determination mode 344a is selected, first, it is determined whether the person within the imaging data that is determined by the person determination unit 342 includes a caregiver or not, as shown in FIG. 15 (S51). In a case where the person within the imaging data includes a caregiver (S51: YES), the assistive task determination unit 344 extracts a movement or shape of a characteristic part of the caregiver, such as a hand area or an arm area, from the imaging data (S52). Also, the gesture data are read from the assistive task determination data storage unit 334 (S53). Next, the movement or shape of the characteristic part is compared with the gesture data, and degree of similarity between patterns are calculated (S54). Subsequently, it is determined whether or not the degree of similarity equal to or greater than the predetermined threshold value exists in the gestures (S55). Accordingly, whether a predetermined gesture performed by the caregiver is included in the imaging data or not is detected. In a case where a gesture with the predetermined threshold value or higher exists (S55: YES), the type of the assistive task associated with the relevant gesture pattern is determined as the type of the assistive task performed by the caregiver (S56). Then, the determined type of the assistive task performed by the caregiver is stored in the care history storage unit 337 along with the state type of the care recipient, as shown in FIG. 5 (S57). Thereafter, the determination of the assistive task ends.

On the other hand, in a case where the gesture with the predetermined threshold value or higher does not exist (S55: NO) and where a caregiver is not included in the person within the imaging data (S51: NO), it is determined that the caregiver does not perform an assistive task, ending the determination of the assistive task.

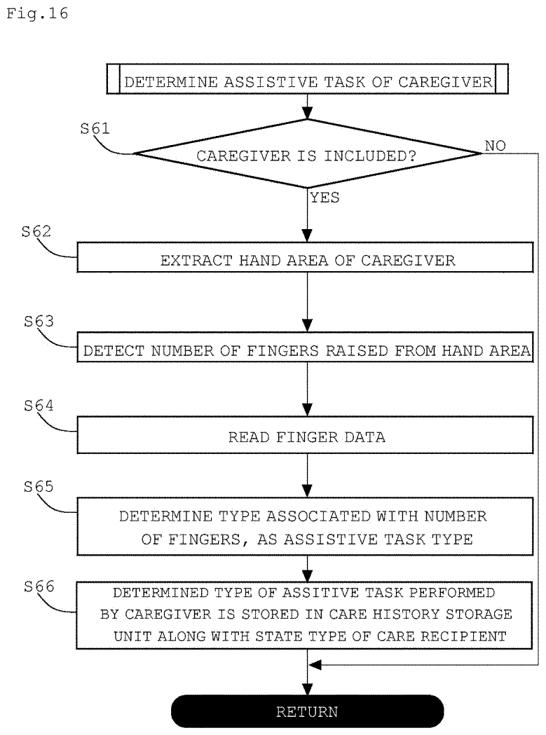

In a case where the finger determination mode 344b is selected, as shown in FIG. 16, it is determined whether the person within the imaging data that is determined by the person determination unit 342 includes a caregiver or not (S61). In a case where a caregiver is included (S61: YES), the hand area of the caregiver is extracted from the imaging data (S62). Then, the number of fingers raised in the extracted hand area is determined (S63). For example, in the present embodiment, the imaging data are binarized into the hand area and other areas, the number of parts protruding in the hand area is detected, and this detected number is determined as the number of raised fingers of the caregiver. Next, the finger data stored in the assistive task determination data storage unit 334 are read (S64). Thereafter, an assistive task associated with the number of fingers determined in S63 is extracted from the finger data, and this assistive task is determined as the type of the assistive task performed by the caregiver (S65). Subsequently, the determined type of the assistive task performed by the caregiver is stored in the care history storage unit 337 along with the state type of the care recipient (S66), as shown in FIG. 5, and then the determination of the assistive task ends.

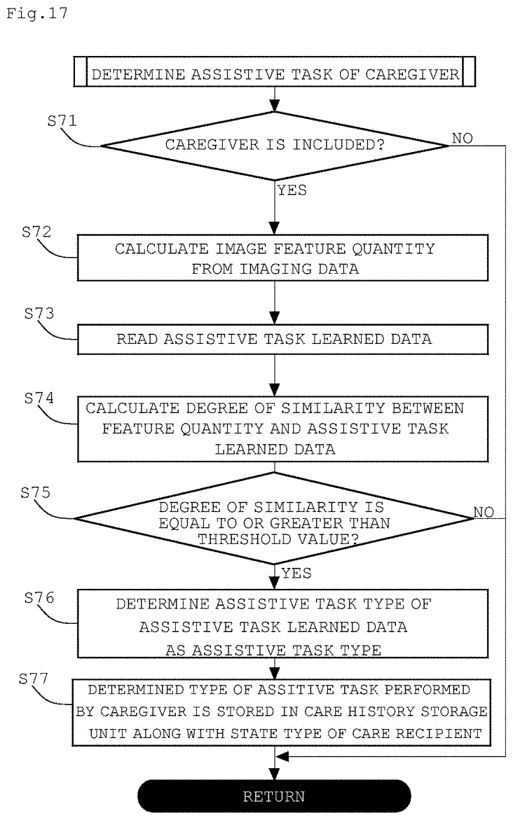

In a case where the assistive task learning determination mode 344c is selected, as shown in FIG. 17, it is determined whether the person within the imaging data that is determined by the person determination unit 342 includes a caregiver or not (S71). In a case where a caregiver is included (S71: YES), the image feature quantity is calculated from the imaging data (S72). Next, the assistive task learned data are read from the assistive task determination data storage unit 334 (S73). Then, the degree of similarity is calculated based on the calculated image feature quantity and each assistive task learned data value (S74). Also, it is determined whether or not the calculated degree of similarity is equal to or greater than the predetermined threshold value (S75). In a case where the degree of similarity is equal to or greater than the predetermined threshold value (S75: YES), the type of the relevant assistive task learned data is determined as the assistive task performed by the caregiver (S76). Subsequently, the determined type of the assistive task performed by the caregiver is stored in the care history storage unit 337 along with the state type of the care recipient (S77), as shown in FIG. 5, and then the determination of the assistive task ends.

Therefore, in the assistive task learning determination mode 344c, the caregiver does not need to make a gesture or raise a finger. Accordingly, the occurrence of record omission due to the caregiver forgetting to record, can be reduced.

Next, the abnormal state assessment unit 346 assesses whether the state of the care recipient is normal or abnormal, based on the physical evaluation level of the care recipient and the type of the state of the care recipient assessed from the imaging data (abnormal state assessment step: S5). In the present embodiment, in a case where the state of the care recipient is determined by the posture determination mode 343a and the state learning determination mode 343b, it is assessed whether the state of the care recipient corresponds to the normal state or the abnormal state that is previously defined in accordance with the combinations of physical evaluation levels and state types of care recipients that are stored in the abnormal state determination data storage unit 336. Furthermore, in a case where the body part area determination mode 343c assesses that the position of the body part matching the body part set by the body part setting unit 322 is located in an area set by the area setting unit 321, information indicating whether the area is an attention-required area or a dangerous area is acquired from the state determination data storage unit 333. In a case where the area is the attention-required area, it is assessed that the care recipient is in a state requiring attention, and in a case where the area is the dangerous area, it is assessed that the care recipient is in a dangerous state.

When it is assessed that the state of the care recipient is an abnormal state, or when the result of determination by the body part area determination mode 343c indicates “state requiring attention” and “dangerous state,” the abnormal state notification unit 347 transmits the abnormality to the display input means 32 or the external communication terminal 4 via the communication means 31 (abnormal state notification step: S6). In the present embodiment, information indicating abnormality, information indicating a state requiring attention and a dangerous state, and the imaging data of the care recipient having abnormality, are transmitted. The display input means 32 or the external communication terminal 4 that receive these information items display, on the display screen, the imaging data of the care recipient in the received abnormal state or having abnormality, and emit a notification sound from the speaker. As a result, the caregiver working in the vicinity of the display input means 32 or the caregiver carrying the external communication terminal 4 can promptly be notified of the abnormal state of the care recipient.

Steps S1 to S6 described above are repeatedly processed while the care histories are recorded.

In a case where the display input means 32 or the external communication terminal 4 instructs the care recording image generation unit 345 to display a care record, the care recording image generation unit 345 generates a care recording image using the care history information and causes the display input means 32 to display the care recording image or transmits the care recording image to the external communication terminal 4 via the communication means 31, as shown in FIG. 8. In the care recording image, since the state types of care recipients and the types of assistive tasks are described side by side on the same screen, a daily life of each care recipient can be displayed so as to be easily understood at a glance. Moreover, since the state types and the types of assistive tasks are uniformly managed in the care history table, a plurality of databases do not need to be read and integrated, enabling easy and efficient display of the state types and the types of assistive tasks. In addition, in a case where displaying of abnormality records is instructed, abnormality recording images are generated using the care history information and displayed by the display input means 32 or transmitted to the external communication terminal 4 via the communication means 31, as shown in FIG. 9. Since the abnormality recording images display the imaging data of abnormal states along with the dates and times thereof, the actual states (postures) can be verified.

According to the care recording device 3, the care recording system 1, the care recording program 3a, and the care recording method of the present embodiment described above, the following effects can be achieved.

1. Since the care recording camera 2 has both the function of inputting the states of care recipients and the function of inputting assistive tasks performed by caregivers, a large number of sensors, terminal devices or the like do not need to be installed, and care histories can be recorded with an inexpensive, simple system configuration.

2. Since the types of states are recorded as care records instead of recording the imaging data (images or videos) obtained by the care recording camera 2, the privacy of the care recipients can be protected.

3. Since assistive tasks performed by caregivers do not need to be directly input using an operation terminal or the like, the load of the recording work can be reduced.

4. Since a care recording image showing the state types of the care recipients and the types of assistive tasks side by side on the same screen can be generated, daily lives of care recipients and whether caregivers perform assistive tasks or not can be verified easily at a glance.

5. Since a normal state and an abnormal state according to the physical evaluation levels of the care recipients can be assessed, quick and detailed assistive tasks can be provided to each care recipient.

Note that the care recording device, the care recording system, the care recording program, and the care recording method according to the present invention are not limited to the embodiment described above and therefore can be changed as appropriate. For example, in the foregoing embodiment, the care recording device 3 has the care recording image generation unit 345, but does not always have to be provided with the care recording image generation unit 345. In addition, the care history information storage unit 337 may transmit data to an external data server or the like to be stored therein. In addition, the person data and the physical evaluation levels may be integrated and stored in the same storage unit.

### REFERENCE SIGNS LIST

1 Care recording system
2 Care recording camera
3 Care recording device
3a Care recording program
4 External communication terminal
31 Communication means
32 Display input means
33 Storage means
34 Arithmetic processing means
321 Area setting unit
322 Body part setting unit
331 Program storage unit
332 Person data storage unit
333 State determination data storage unit
334 Assistive task determination data storage unit
335 Physical evaluation level storage unit
336 Abnormal state determination data storage unit
337 Care history storage unit
341 Imaging data acquisition unit
342 Person determination unit
343 Care recipient state determination unit
343a Posture determination mode
343b State learning determination mode
343c Body part area determination mode
344 Assistive task determination unit
344a Gesture determination mode
344b Finger determination mode
344c Assistive task learning determination mode
345 Care recording image generation unit
346 Abnormal state assessment unit
347 Abnormal state notification unit

1. A care recording device for recording a state of a care recipient and an assistive task provided for the care recipient by a caregiver, the care recording device comprising:
an imaging data acquisition unit that acquires imaging data from a camera capturing an image of the care recipient;
a person determination unit that detects a person imaged, based on the imaging data, to determine whether the person is the care recipient or the caregiver;
a care recipient state determination unit that determines a state type of the care recipient based on the imaging data, associates the state type including a normal state with dates and times consecutive at predetermined time intervals, and stores the association as care history information in a care history storage unit; and
an assistive task determination unit that, in a case where the person determined by the person determination unit includes the caregiver, determines a type of the assistive task of the caregiver based on the imaging data, associates the type of the assistive task with the care history information, and stores the association in the care history storage unit.

2. The care recording device according to claim 1, wherein the care recipient state determination unit has a posture determination mode for detecting each coordinate of a body part representing a posture of the care recipient from the imaging data, and determining the state type of the care recipient based on the each coordinate of the body part.

3. The care recording device according to claim 1, wherein the care recipient state determination unit has a state learning determination mode for determining the state type based on state learned data obtained by learning the imaging data obtained in advance for each state of the care recipient and the imaging data acquired.

4. The care recording device according to claim 1, further comprising:
an area setting unit that sets an area smaller than an imaging range of the imaging data within the imaging range; and
a body part setting unit that sets the body part of the care recipient in association with the area set by the area setting unit,
wherein the care recipient state determination unit has a body part area determination mode that detects, from the imaging data, a position within the imaging range representing each of body parts of the care recipient, and determines the state type of the care recipient based on whether or not a position of a body part set by the body part setting unit out of positions of the body parts exists within the area set by the area setting unit.

5. The care recording device according to claim 1, wherein the assistive task determination unit has a gesture determination mode for detecting, from the imaging data, whether the caregiver makes a predetermined gesture or not, and when the gesture is detected, determining a type of the assistive task associated with the gesture.

6. The care recording device according to claim 1, wherein the assistive task determination unit has a finger determination mode for detecting, from the imaging data, whether the caregiver makes a finger raising movement or not, and when the finger raising movement is detected, determining a type of the assistive task associated with the number of fingers raised.

7. The care recording device according to claim 1, wherein the assistive task determination unit has an assistive task learning determination mode for determining a type of the assistive task based on assistive task learned data acquired by learning the imaging data obtained in advance for each type of the assistive task, and the imaging data acquired.

8. The care recording device according to claim 1, further comprising a care recording image generation unit that uses the care history information stored in the care history storage unit to generate a care recording image for displaying the state type of the care recipient and the type of the assistive task side by side on a same screen, for each care recipient.

9. The care recording device according to claim 1, further comprising:
a physical evaluation level storage unit that stores a physical evaluation level defined for each care recipient;
an abnormal state assessment unit that assesses whether or not a state type of a target care recipient determined by the care recipient state determination unit is an abnormal state corresponding to the physical evaluation level of the care recipient; and
an abnormal state notification unit that, when the care recipient is assessed to be in an abnormal state, notifies that the care recipient is in the abnormal state.

10. A care recording system, comprising:
the care recording device according to claim 1; and
a care recording camera that is installed in a living room of the care recipient, captures an image of the room, and transmits resultant imaging data to the care recording device.

11. A care recording program for recording a state of a care recipient and an assistive task provided for the care recipient by a caregiver, the care recording program causing a computer to function as:
an imaging data acquisition unit that acquires imaging data from a camera capturing an image of the care recipient;
a person determination unit that detects a person imaged, based on the imaging data, to determine whether the person is the care recipient or the caregiver;
a care recipient state determination unit that determines a state type of the care recipient based on the imaging data, associates the state type including a normal state with dates and times consecutive at predetermined time intervals, and stores the association as care history information in a care history storage unit; and
an assistive task determination unit that, in a case where the person determined by the person determination unit includes the caregiver, determines a type of the assistive task of the caregiver based on the imaging data, associates the type of the assistive task with the care history information, and stores the association in the care history storage unit.

12. A care recording method for recording a state of a care recipient and an assistive task provided for the care recipient by a caregiver, the care recording method comprising:
an imaging data acquisition step of acquiring imaging data from a camera capturing an image of the care recipient;
a person determination step of detecting a person imaged, based on the imaging data, to determine whether the person is the care recipient or the caregiver;
a care recipient state determination step of determining a state type of the care recipient based on the imaging data, associating the state type including a normal state with dates and times consecutive at predetermined time intervals, and storing the association as care history information in a care history storage unit; and
an assistive task determination step of, in a case where the person determined by the person determination step includes the caregiver, determining a type of the assistive task of the caregiver based on the imaging data, associating the type of the assistive task with the care history information, and storing the association in the care history storage unit.
